(19) United States
(12) Patent Application Publication
(10) Pub. No.: US 2006/0259056 A1
(43) Pub. Date: Nov. 16, 2006

(54) Surgical holder for a blood vessel

(76) Inventors: Go Watanabe, Ishikawa (JP); Yoshiki Sawa, Hyogo (JP); Satoshi Taketani, Osaka (JP)

Correspondence Address: SNELL & WILMER, 400 EAST VAN BUREN, ONE ARIZONA CENTER, PHOENIX, AZ, 85004-2202, US

(21) Appl. No.: 10/550,819

(22) PCT Filed: Mar. 23, 2004
(86) PCT No.: PCT/IB04/50314

§ 371 Date: Jun. 21, 2006

(30) Foreign Application Priority Data
Mar. 25, 2003 (JP) 2003-082438
Sep. 25, 2003 (JP) 2003-334379

(51) Int. Cl. A61B 17/32

(52) U.S. Cl. 606/181

(57) ABSTRACT

A surgical holder comprises a grasping member for grasping a tissue, a manipulation member for manipulating the grasping member, and a connection portion with one end connected to the manipulation member, wherein: the grasping member includes a first grasping plate, and a second grasping plate provided so as to oppose the first grasping plate in a movable manner so that they are able to become closer to each other or more distranced from each other; a first grasping portion which can grasp a part of a tissue between the first grasping plate and the second grasping plate, and an opening which can expose another part of the tissue are provided in one end portion of the grasping member; and a second grasping portion which can form a tissue grasping space between the first grasping plate and the second grasping plate is provided in another end portion of the grasping member.

### TECHNICAL FIELD

The present invention relates to a surgical holder, particularly, a surgical holder for blood vessel surgery which is suitable for coronary bypass surgery and the like.

### BACKGROUND ART

Conventionally, bypass surgery using heart-lung machines has been generally conducted for patients suffering myocardial infarction or angina pectoris. When such surgery is conducted, extracorporeal circulation accompanied with the use of heart-lung machines causes complication to occur at a certain ratio, and affects prognosis of the patient.

In recent years, surgical operations have increasingly become less invasive. In the field of cardiac surgery, bypass surgery on a beating heart which does not employ heart-lung machines has become widely used. In bypass surgery on a beating heart, while a removed artery or vein is being connected to the heart, generally, an assistant holds part of a blood vessel of the patient with tweezers.

Grasping a tissue such as a blood vessel with tweezers requires skill. When a very delicate tissue is grasped, the tissue may be undesirably damaged. Further, there is a problem that a sufficient view is required for manipulation in surgery, the view may be obstructed by an operation of holding a blood vessel. Conventionally, a tool for grasping a blood vessel has been developed (see, Japanese Utility Model Laid-Open Publication No. 7-17208), but further improvement is demanded in terms of manipulability, stability in grasping, applicability, and the like. Furthermore, a tool which may be used for manipulating the grasped tissue such as during anastomosis manipulation, a tool having a multifunction which may by itself address various manipulations during surgery, and the like has been strongly desired.

The objective of the present invention is to provide a tool which can stably grasp a tissue such as a blood vessel without causing damage and which enables various manipulations during surgery,

In view of the problems of the conventional art, the present inventors have conducted a diligent study. As a result, the present inventors have found that a tool having a specific structure well adapted to manipulations during surgery, and achieved the present invention through further study.

### DISCLOSURE OF THE INVENTION

The present invention is described as follows.

1. A surgical holder comprising a grasping member for grasping a tissue, a manipulation member for manipulating the grasping member, and a connection portion with one end connected to the manipulation member, wherein:

the grasping member includes a first grasping plate, and a second grasping plate providedso as to oppose the first grasping plate in a movable manner so that they are able to become closer to each other or more distanced from each other;

a first grasping portion which can grasp a part of a tissue between the first grasping plate and the second grasping plate, and an opening which can expose another part of the tissue when the part of the tissue is grasped by the first grasping portion are provided in one end portion of the grasping member; and

a second grasping portion which can form a tissue grasping space between the first grasping plate and the second grasping plate is provided in another end portion of the grasping member.

2. A surgical holder according to item 1, wherein a tissue protection material is attached to an opposing surface of the first grasping plate andlor the second grasping plate.

3. A surgical holder according to item 2, wherein the tissue protection material is permeated with medicines.

4. A-surgical holder comprising a grasping member for grasping a tissue, a manipulation member for manipulating the grasping member, and a connection portion with one end connected to the manipulation member and the other end provided with a fixing tool, wherein:

the grasping member includes a first grasping plate, and a second grasping plate provided so as to oppose the first grasping plate in a movable manner so that they are able to become closer to each other or more distanced from each other;

the first grasping plate includes a retaining portion having the opening and a supporting portion having a recessed portion;

the second grasping plate includes a covering portion formed so as to cover an entire surface or a part of the opening of the first grasping plate, a non-covering portion which does not cover the first grasping plate, and a fixing portion which is provided so as to oppose the recessed portion of the first grasping plate and has a curved portion which can form a tissue grasping space.

5. A surgical holder comprising a grasping member for grasping a tissue, a manipulation member for manipulating the grasping member, and a connection portion with one end connected to the manipulation member and the other end provided with a fixing tool, wherein:

the grasping member includes a first grasping plate, and a second grasping plate provided so as to oppose the first grasping plate in a movable manner so that they are able to become closer to each other or more distanced from each other;

the grasping member is formed into a rectangular shape with the manipulation member elongated from a side thereof;

a first grasping portion which can grasp a part of a tissue between the first grasping plate and the second grasping plate, and an opening which is provided in the vicinity of the first grasping portion and exposes another part of the tissue are provided in one end portion of the grasping member; and

a second grasping portion which can form a tissue grasping space between the first grasping plate and the second grasping plate is provided in another end portion of the grasping member.

6. A surgical holder comprising a grasping member for grasping a tissue, a manipulation member for manipulating the grasping member, and a wiring portion connected to the manipulation member, wherein:

the grasping member includes a first grasping plate and a second grasping plate;

the first grasping plate includes a retaining portion having an opening of a U-shape or substantially a U-shape, and a supporting portion having a recessed portion; and

the second grasping plate includes a covering portion formed to cover an entire surface or a part of the opening of the first grasping plate, a non-covering portion which does not cover the first grasping plate, and a fixing portion having a curved portion.

7. A surgical holder according to item 6, wherein the tissue to be grasped is a tubular tissue with a surrounding tissue, and the surrounding tissue is grasped by a part which defines the shape of the opening of the first grasping plate and the covering portion of the second grasping plate.

8. A surgical holder according to item 6, wherein the tissue to be grasped is a tubular tissue, and the tubular tissue is grasped by a tissue grasping space formed by the recessed portion of the first grasping plate and the curved portion of the second grasping plate.

9. A surgical holder according to item 6, wherein the tissue to be grasped is a tubular tissue, and the tubular tissue is grasped with one point of the tubular tissue being grasped by the retaining portion of the first grasping plate and the covering portion of the second grasping plate, and another point being grasped by a tissue grasping space formed by the recessed portion of the first grasping plate and the curved portion of the second grasping plate.

10. A surgical holder according to item 6, wherein the tissue to be grasped is a tubular tissue, and an end portion of the retaining portion which defines the opening of the first grasping plate is inserted into a tube of the tubular tissue to grasp the tubular tissue.

11. A surgical holder according to item 6, wherein the tissue to be grasped is a tubular tissue (A), and, (i) with one point of the tubular tissue (A) grasped by the retaining portion of the first grasping plate and the covering portion of the second grasping plate and another point of the tubular tissue (A) grasped by the tissue grasping space formed by the recessed portion of the first grasping plate and the curved portion of the second grasping plate, an astomosis manipulation between a cut portion of thetubular tissue (A) positioned on the supporting portionand a cut portion of a tubular tissue (B) is performed, and then, (ii) with the tubular tissue (A) grasped by the tissue grasping space formed by the recessed portion of the first grasping plate and the curved portion of the second grasping plate, an astomosis manipulation between a cut portion of the tubular tissue (A) positioned on the supporting portionand a cut portion of the tubular tissue (C) is performed.

The surgical holder of the present invention is preferably a surgical holder for blood vessel surgery, and more preferably, a surgical holder for coronary bypass surgery.

Furthermore, the surgical holder of the present invention is preferably a surgical holder in which a tubular tissue to be grasped is a blood vessel.

The surgical holder of the present invention can grasp and fix tissues of various forms and can preferably grasp blood vessel tissues and the like including almost all the types of artery and vein. For example, it is possible to grasp a tissue removed with a surrounding tissue thereof or to grasp a skeletonized tissue.

Furthermore, the skeletonized tissue can be grasped at a plurality of points at one time. Moreover, a further manipulation on the grasped tissue such as a sequential grafting or the like can be performed. The first grasping plate and the second grasping plate can be used as a supporting platform or a manipulation platform to improve stability of manipulation.

Further, the surgical holder of the present invention has a tissue protection material 10 having a slip stopping function and a tissue protection function. Thus, a tissue can be stably grasped without damaging the tissue.

Further, it is possible to permeate the tissue protection material with medicines and appropriately perform treatments such as preventing a tissue from being dried or preventing cell injury.

Further, the holder has a wiring portion, and it is possible to freely set the position of a grasping member 1 and a manipulation member 2. Moreover, it is possible to enhance convenience of an operator by fixing another end of the wiring portion to a rib spreader or the like.

Conventionally, a tissue was held by an assistant or the like during surgery. According to the holder of the present invention, someone's assistance in grasping is not required. Moreover, the problem that a person's hand obstructs the view can be solved.

Furthermore, since the holder of the present invention has a compact structure, a cut portion in surgery can be made smaller, and thus, damage on a body of a patient can be reduced. The holder of the present invention has a simple structure and can be made of a widely used material such as a resin. Thus, a cost can also be reduced.

Moreover, the holder of the present invention is a multifunctional tool which can address various operations by itself. Thus, there is no need to use a plurality of tools and surgical manipulations become more convenient and efficient.

The present invention with the above-described advantages has particularly superior effects in less invasive surgery, of which the number of applied cases is predicted to increase, and thus, is useful.

### BRIEF DESCRIPTION OF THE DRAWINGS

FIG. 1 shows an exemplary grasping member, manipulation member, connection portion and wiring portion of the holder of the present invention;

FIG. 2 is a plan view of an exemplary grasping member and manipulation member of the holder of the present invention when viewed from the side of a first grasping plate;

FIG. 3 is a plan view of an exemplary grasping member and manipulation member of the holder of the present invention when viewed from the side of a second grasping plate;

FIG. 4 is a schematic view of a part of the manipulation of the holder of the present invention with arrows indicating a direction of force applied to the manipulation member and a direction of movement of the grasping member in co-operation with the movement of the manipulation member;

FIG. 5 is a view of an exemplary grasping member and the manipulation member of the holder of the present invention when viewed from the side of the opening;

FIG. 6 is a view of an exemplary grasping member and the manipulation member of the holder of the present invention when viewed from the side of a recessed portion of the first grasping plate and a curved portion of the second grasping plate;

FIG. 7 is a schematic view of a part of the manipulation of the holder of the present invention when viewed from the side with arrows indicating a direction of force applied to the manipulation member and a direction of movement of the grasping member in cooperation with the movement of the manipulation member;

FIG. 8 is a schematic view showing a tubular tissue being grasped with a surrounding portion thereof by the holder of the present invention;

FIG. 9 is a schematic view showing a skeletonized tubular tissue being grasped by the holder of the present invention;

FIG. 10 is a schematic view showing a skeletonized tubular tissue being grasped at two points by the holder of the present invention;

FIG. 11 is a schematic view showing a tubular tissue being grasped by the holder of the present invention;

FIG. 12 is a schematic view showing sequential grafting being performed by using the holder of the present invention;

FIG. 13 is a plan view of another exemplary grasping member and manipulation member of the holder of the present invention when viewed from the side of a first grasping plate;

FIG. 14 is a plan view of another exemplary grasping member and manipulation member of the holder of the present invention when viewed from the side of a second grasping plate;

FIG. 15 is a plan view of yet another exemplary grasping member and manipulation member of the holder of the present invention when viewed from the side of a first grasping plate;

FIG. 16 is a plan view of yet another exemplary grasping member and manipulation member of the holder of the present invention when viewed from the side of a second grasping plate;

FIG. 17 is a plan view of still another exemplary grasping member and manipulation member of the holder of the present invention when viewed from the side of a first grasping plate;

FIG. 18 is a plan view of still another exemplary grasping member and manipulation member of the holder of the present invention when viewed from the side of a second grasping plate; and

FIGS. 19A through 19E are schematic views of various shapes of a tissue grasping space formed between the first grasping plate and the second grasping plate.

### BEST MODE FOR CARRYING OUT THE INVENTION

The embodiments of the present invention will be specifically described below.

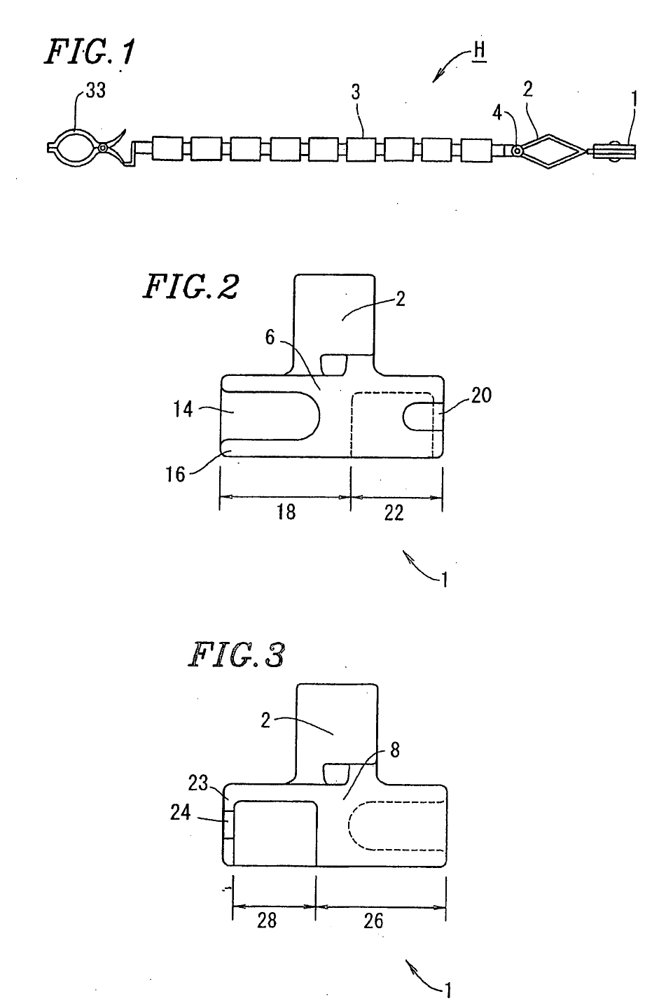

As shown in FIG. 1, a surgical holder H according to the present invention includes a grasping member 1 for grasping a tissue, manipulation member 2 for manipulating the grasping member 1, and a wiring portion 3 connected to the manipulation member 2, which is a coupling portion. The holder H according to the present invention may further include a connection portion 4 for connecting the wiring portion 3 and the manipulation member 2.

(Grasping Member 1)

The grasping member 1 is a part for grasping a tissue and includes a first grasping plate 6 and a second grasping plate 8.

The first grasping plate 6 and the second grasping plate 8 can be overlaid and oppose each other. A tissue inserted between the first grasping plate 6 and the second grasping plate 8 is pinched and grasped with opposing inner side surfaces of the first grasping plate 6 and the second grasping plate 8. More specifically, the second grasping plate 8 is provided so as to oppose the first grasping plate 6 in a movable manner so that they are able to become closer to each other or more distanced from each other.

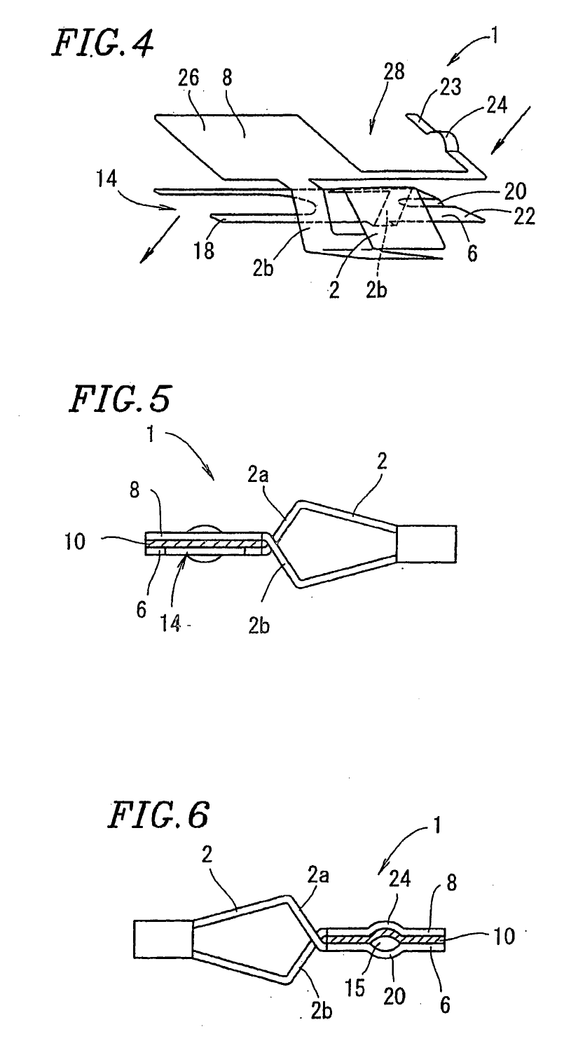

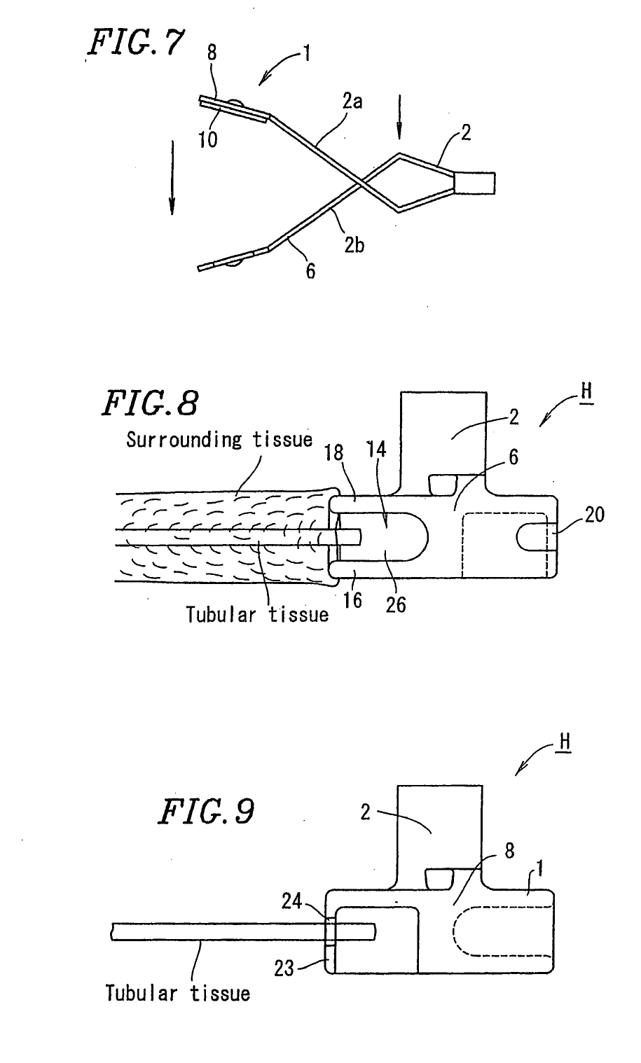

As shown in FIG. 2 through 7, the first grasping plate 6 includes a retaining portion 18 having an opening 14 of a U-shape or substantially a U-shape, and a supporting portion 22 having a recessed portion 20. The peripheral portion of the retaining portion 18 except for the opening 14 and the supporting portion 22 are continuous.

The retaining portion 18 of the first grasping plate 6 pinches a tissue with a covering portion 26 of the second grasping plate 8 and retains the tissue. The retaining portion 18 has the opening 14 of a U-shape or substantially a U-shape and an edge portion 16 which defines the shape of the opening 14.

The opening 14 is a notch portion of a U-shape or substantially a U-shape which is located in an end portion of the first grasping plate 6. The term ‘substantially a U-shape’ means a shape approximately the same as a U-shape and includes, for example, a semiellipse shape, and a semi-quadrilateral shape. The edge portion 16 which defines the shape of the opening 14 is the peripheral portion of the opening 14. End portions of the edge portion 16 which defines the shape of the opening 14 define upper edges of the ‘u’ in the U-shape or substantially U-shape of the opening 14 and are elongated.

In other words, a first grasping portion which can grasp a part of a tissue between the first grasping plate 6 and the second grasping plate 8 and the opening 14 which may expose another part of the tissue when the part of the tissue is grasped by the first grasping portion are provided in one end portion of the grasping member 1. Further, a second grasping portion which may form a tissue grasping space 15 between the first grasping plate 6 and the second grasping plate 8 is provided in the other end portion of the grasping member 1. The first grasping portion includes the retaining portion 18 of the first grasping plate 6 and the covering portion 26 of the second grasping plate 8. The second grasping portion includes the recessed portion 20 of the first grasping plate 6 and a curved portion 24 of the second grasping plate 8.

The above-described structure enables an appropriate grasping of a tissue. For example, if a tissue to be grasped is a tubular tissue and a surrounding tissue thereof, the surrounding tissue is pinched by the retaining portion 18 which defines the shape of the opening 14 of the first grasping plate 6 and the covering portion 26 of the second grasping plate 8 to position the tubular tissue at the opening 14. Thus, it is possible to grasp a surrounding tissue without directly retaining a tubular tissue which is susceptible to damage. Further, if a tissue to be grasped is a tubular tissue which has a stump, the end portion of the edge portion 16 of the retaining portion 18 which defines the shape of the opening 14 of the first grasping plate 6 is inserted into a tube of the tubular tissue to grasp the tubular tissue so the tissue hangs suspended from a tip portion of the edge portion 16.

The supporting portion 22 of the first grasping plate 6 supports the tissue being grasped, and has the recessed portion 20 in the end portion of the supporting portion 22. The recessed portion 20 of the supporting portion 22 and the opening 14 of the retaining portion 18 are preferably positioned at both ends of one straight line. The recessed portion 20 forms a pair with the curved portion 24 of the second grasping plate 8 to form the tissue grasping space 15. A shape of the recessed portion 20 is not specifically limited. However, the recessed portion 20 is preferably a recess of a semicircle.

In addition to the retaining portion 18, the supporting portion 22 also enables appropriate grasping of a tissue. For example, if a tissue to be grasped is a tubular tissue, the tubular tissue is grasped by the retaining portion 18 of the first grasping plate 6 and the covering portion 26 of the second grasping plate 8 at one point, and the tubular tissue is grasped by the tissue grasping space 15 formed by the recessed portion 20 of the first grasping plate 6 and the curved portion 24 of the second grasping plate 8 at another point. By grasping the tubular tissue at a plurality of points, it is possible to grasp the tissue more stably.

Furthermore, the supporting portion 22 may be used as a manipulation platform for further manipulation or processing of the grasped tissue. For example, in the case where the tubular tissue is grasped at at least two points, the tubular tissue between the two points is positioned on the supporting portion 22. Thus, manipulation such as anastomosis manipulation or the like can be performed on the supporting portion 22.

A tissue protection material 10 to be attached to the first grasping plate 6 and/or the second grasping plate 8 is formed so as to position between the first grasping plate 6 and the second grasping plate 8. A range to be covered by tissue protection material 10 may be an entire surface or a part of the first grasping plate 6 and/or the second grasping plate 8. However, the tissue protection material 10 is preferably attached to the entire surface on the side of the second grasping plate 8 which opposes the first grasping plate 6.

The tissue protection material 10 has both a tissue protection function and a slip stopping function. The tissue protection material 10 allows the prevention of damage to the tissue to be grasped and a more stable grasping of the tissue. The tissue protection material 10 may be permeated with various medicines, or subjected to a surface process to give additional functions to the surgical holder H.

The tissue protection material 10 is preferably flexible, and, for example, sponge or the like may be suitably used. The type of base material used for the tissue protection material 10 is not particularly limited. However, it is preferable to be a hydrophilic base material. If a hydrophilic base material is used, it becomes easy to have the tissue protection material 10 permeated with a solution including various medicines to give functions such as preventing a tissue from being dried or preventing cell injury. The medicines permeating through the tissue protection material 10 may be, for example, an anticoagulant agent, a vasodilator, a blood vessel growth promotion factor, various proteins, an immunosuppressive agent, and the like.

The second grasping plate 8 includes a covering portion 26 which is formed to cover an entire surface or a part of the opening 14 of the first grasping plate 6, a noncovering portion 28 which does not cover the first grasping plate 6, and a fixing portion 23 having the curved portion 24. More specifically, the second grasping plate 8 includes the fixing portion 23 provided to extend from the covering portion 26, which is formed so as to cover an entire surface or a part of the opening 14 of the first grasping plate 6, through an elongated piece, and includes the non-covering portion 28 with an opening of a rectangular shape formed by an edge of the covering portion 26, the elongated piece, and the fixing portion 23.

The covering portion 26 of the second grasping plate 8 covers an entire surface or a part of the opening 14 of the first grasping plate 6, when the first grasping plate 6 and the second grasping plate 8 are manipulated so as to oppose each other and overlay. It is preferable that the covering portion 26 is formed so as to cover the entire surface of the opening 14, and it is further preferable that the covering portion 26 is formed so as to cover not only the opening 14, but also surrounding portions of the opening 14. Particularly, when the first grasping plate 6 and the second grasping plate 8 are manipulated so as to oppose each other and overlay, it is preferable that the covering portion 26 is formed so as to cover the retaining portion 18 of the first grasping plate 6.

The covering portion 26 pinches a tissue with the retaining portion 18 of the first grasping plate 6 to grasp the tissue. Further, the covering portion 26 supports or fixes the grasped tissue to improve the stability. For example, if a tissue to be grasped is a tubular tissue and the stir-rounding tissue thereof and the surrounding tissue is pinched by the retaining portion 18 which defines the shape of the opening 14 of the first grasping plate 6 and the covering portion 26 of the second grasping plate 8, the tubular tissue positioned at the opening 14 is supported by the covering portion 26. Further, if a tissue to be grasped is a tubular tissue which has a stump and the end portion of the edge portion 16 of the retaining portion 18 which defines the shape of the opening 14 of the first grasping plate 6 is inserted into a tube of the tubular tissue to grasp the tubular tissue so the tissue hangs suspended from a tip portion of the edge portion 16, the tubular tissue can be grasped more stably by pressing the side surface of the tubular tissue grasped by the covering portion 26.

The non-covering portion 28 of the second grasping plate 8 does not cover the first grasping plate 6 when the first grasping plate 6 and the second grasping plate 8 are manipulated so as to oppose each other and overlay. The non-covering portion 28 approximately corresponds to the supporting portion 22 of the first grasping plate 6 when the first grasping plate 6 and the second grasping plate 8 oppose each other.

The non-covering portion 28 enables a manipulation or process with the surgical holder H grasping a tissue. For example, when the retaining portion 18 of the first grasping plate 6 and the covering portion 26 of the second grasping plate 8 grasp a tubular tissue at one point, the tissue grasping space 15 which is formed with the recessed portion 20 of the first grasping plate 6 and the curved portion 24 of the second grasping plate 8 grasps the tubular tissue at another point, and a middle portion of the tubular tissue between the two points is positioned on the supporting portion 22, it becomes possible to appropriately perform manipulation or processing of the tissue on the supporting portion 22 from the side of the opening of the non-covering portion 28.

The fixing portion 23 of the second grasping plate 8 has a curved portion 24 and fixes the tissue. The fixing portion 23 and the covering portion 26 are preferably positioned at respective ends on one axis of the second grasping plate 8 with the noncovering portion 28 being therebetween.

The curved portion 24 opposes the recessed portion 20 of the first grasping plate 6 to form the tissue grasping space 15 when the first grasping plate 6 and the second grasping plate 8 are positioned so as to oppose one another. The shape of the tissue grasping space 15 is not specifically limited. However, it is preferable to form a substantially circular space 15. The tissue grasping space 15 enables appropriate grasping of, for example, a skeletonized tubular tissue without cutting it.

The material to form the first grasping plate 6 and the second grasping plate 8 is not specifically limited. Various types of polymer, such as polypropylene, acrylonitrile butadiene styrene copolymer (ABS), polymethyl pentene, and the like may be used. The first grasping plate 6 and the second grasping plate 8 may be transparent substrates.

The size of the first grasping plate 6 and the second grasping plate 8 is not specifically limited and can be appropriately set depending on the types of tissue to be grasped. For example, if the tissue to be grasped is a blood vessel, the grasping member 1 may have a length of about 20 to 40 mm and a width of about 10 to 20 mm, and preferably, a length of about 25 to 35 mm and a width of about 12 to 15 mm.

(Manipulation Member 2)

The manipulation member 2 performs a manipulation of the grasping member 1 in a movable manner so that plates of the grasping member 1 are able to become closer to each other or more distanced from each other. More specifically, the manipulation member 2 performs manipulation to move the first grasping plate 6 and the second grasping plate 8 and pinch the tissue inserted between the first grasping plate 6 and the second grasping plate 8 from both sides.

The structure of the manipulation member 2 may be appropriately designed as desired. For example, the manipulation member 2 may be formed integrally with the grasping member 1 such that the first grasping plate 6 and the second grasping plate 8 move in cooperation with the manipulation member 2 by manipulating the manipulation member 2.

More specifically, the manipulation member 2 may have a structure in which two manipulation plates, a manipulation plate 2a and a manipulation plate 2b form the manipulation member 2 and are integrally formed with the first grasping plate 6 and the second grasping plate 8, respectively, and the manipulation member 2 moves such that the first grasping plate 6 and the second grasping plate 8 are overlaid or separated by applying or removing force to of from the manipulation plates 2a and 2b, or adjusting the force to the manipulation plates 2a and 2b.

The manipulation member 2 may include an adjusting mechanism for applying force in a direction such that the pair of the first grasping plate 6 and the second grasping plate 8 overlay or applying force in a direction such that the first grasping plate 6 and the second grasping plate 8 move back to their original positions. Such an adjusting mechanism may be an elastic body or a spring.

The manipulation member 2 preferably has a structure such that it can give force sufficient for stably grasping a tissue and which does cause damage to the tissue.

(Wiring Portion or Connection Portion 3)

The wiring portion 3 connects the grasping member 1 or the manipulation member 2 to another tool (for example, an arm of a stabilizer) or manipulation means.

The structure of the wiring portion 3 is not specifically limited. However, a flexible wire having a flexibility which allows the operator to freely bend the wire with one hand is preferable. For example, it may be a wire having a flexible backbone structure. The wire may be subjected to a process such as dying, vinyl-coating or the like as appropriate.

The material of the wiring portion 3 is not specifically limited and can be appropriately set as desired. For example, various types of metals such as iron, aluminum, copper, brass and the like, or various types of plastic may be used.

One end of the wiring portion 3 may be connected to the manipulation member 2. The structure of the connection point may be appropriately designed. For example, the wiring portion 3 may be connected to the manipulation member 2 via a suitable connection portion 4. Alternatively, the wiring portion 3 may be integrally formed with the manipulation member 2.

In the case where the wiring portion 3 is connected to the manipulation member 2 via the connection portion 4, the structure of the connection portion 4 may be appropriately designed as desired. Particularly, a structure in which the wiring portion 3 can be moved as freely as possible with the connection portion 4 being a fulcrum is preferable. The specific structure of the connection portion 4 may be a structure which includes a rotation axis which moves in cooperation with the wiring portion 3 and a bearing for supporting the rotation axis which is fixed to the manipulation member 2, and in which wire is connected so as to rotate freely with the rotation axis as a fulcrum.

The structure of the other end of the wiring portion 3 may also be appropriately designed as desired. It may include, for example, a fixing tool 33 for fixing to a rib spreader. The fixing tool may be, for example, a clip.

(A Method for Using the Surgical Holder H)

The surgical holder H of the present invention can be used for grasping or manipulating tissue during surgery.

A tissue to be grasped is not specifically limited. The tissue may be, for example, a tubular tissue or linear tissue, such as a blood vessels, bowel, nerve, etc., and a surrounding tissue thereof. Particularly, the surgical holder H of the present invention

is suitable for grasping blood vessel tissues, such as arteries, veins, and the like.

The surgical holder H of the present invention may be preferably used for surgery, especially, blood vessel surgery. Particularly the surgical holder H may be preferably used for grasping various types of blood tissues in any types of bypass surgery. For example, it is possible to grasp and fix almost all types of artery, such as internal mammary artery (IMA) which is used most frequently as an artery graft for coronary bypass surgery, gastroepiploic arteries (GEA), inferior epigastric artery (IEA), radial artery (RA), and the like. It is also possible to grasp and fix veins such as the large saphenous vein (SV).

Furthermore, the surgical holder H of the present invention can grasp tissues of various forms such as, a tissue having both a tubular tissue and a surrounding tissue, a skeletonized tissue, and the like. For example, according to the present invention, it is possible to grasp an artery which is removed with the surrounding tissue as a so-called pedicle. Further, it is possible to grasp the skeletonized artery.

Further, the grasped tissue may be used for manipulating, sequential grafting or the like.

Specifically, the surgical holder H of the present invention may be used as described below.

One example is a method of use for grasping a tubular tissue or a surrounding tissue of a linear tissue with the retaining portion 18 for defining a shape of the opening 14 of the first grasping plate 6 and the covering portion 26 of the second grasping plate S, as shown in FIG. 8. This method can be applied to the case in which, for example, an arterial graft removed with the surrounding tissue is grasped as a pedicle.

The second example is a method of use for grasping a tubular tissue or a linear tissue with the tissue grasping space 15 formed by the recessed portion 20 of the first grasping plate 6 and the curved portion 24 of the second grasping plate 8, as shown in FIG. 9. This method can be applied to the case in which, for example, a skeletonized artery graft is grasped.

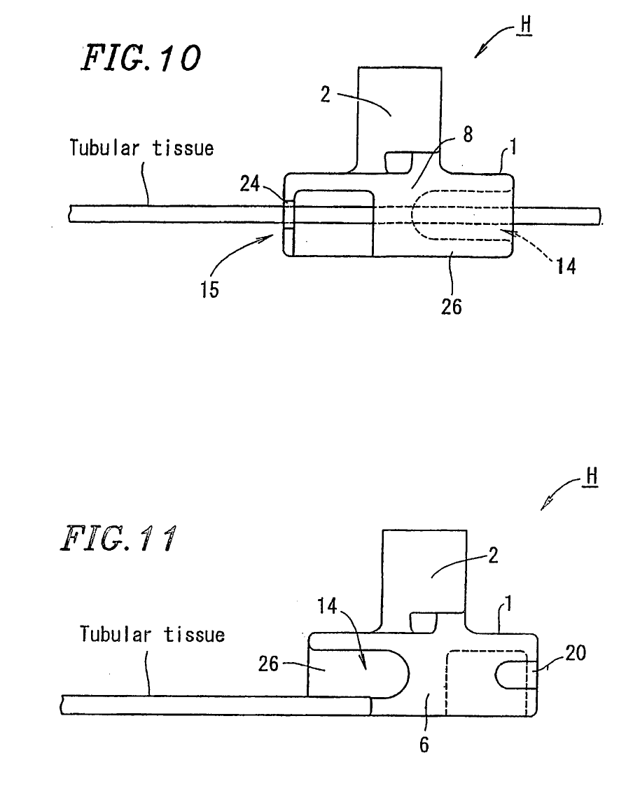

The third example is a method of use for grasping a tubular tissue or a linear tissue at at least two points with the retaining portion 18 of the first grasping plate 6 and the covering portion 26 of the second grasping plate 8 grasping the tubular tissue or the linear tissue at one point, and with the tissue grasping space 15 formed by the recessed portion 20 of the first grasping plate 6 and the curved portion 24 of the second grasping plate 8 grasping the tubular tissue or the linear tissue at another point as shown in FIG. 10. This method can be applied to the case in which, for example, a skeletonized artery graft is grasped at two points.

The fourth example is a method of use for grasping a tubular tissue by inserting the end portion of the part which defines the shape of the opening 14 into a tube of the tubular tissue, as shown in FIG. 11. This method can be applied to the case in which, for example, a veinous graft is grasped.

Furthermore, the surgical holder H of the present invention may be used to perform the sequential grafting described below. Herein, cut portion means a portion where the tissue is incised or amputated, and it refers to, for example, an incised portion formed by a midline incision into a side surface of the tubular tissue, and/or a stump portion which locates at the end portion of the tubular tissue.

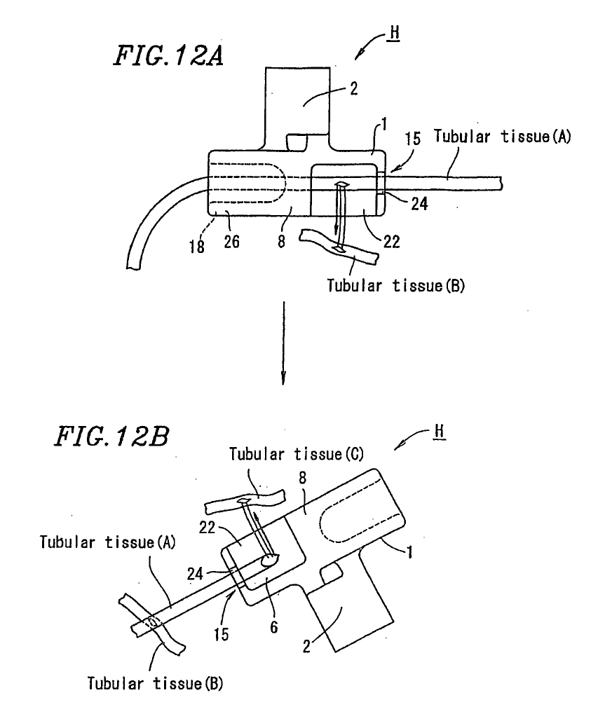

First, as shown in FIG. 12A, the surgical holder H of the present invention is used to grasp a point of a tubular tissue (A) by the retaining portion 18 of the first grasping plate 6 and the covering portion 26 of the second grasping plate 8 and grasp another point of the tubular tissue (A) by the tissue grasping space 15 formed by the recessed portion 20 of the first grasping plate 6 and the curved portion 24 of the second grasping plate 8 and to perform an anastomosis manipulation of a cut portion of the tubular tissue (A) and a cut portion of a tubular tissue (B) positioned on the supporting portion 22. At the same time, as shown in FIG. 12B, the tubular tissue (A) is grasped with the tissue grasping space 15 formed by the recessed portion 20 of the first grasping plate 6 and the curved portion 24 of the second grasping plate 8 to perform an anastomosis manipulation of a cut portion of the tubular tissue (A) and a cut portion of a tubular tissue (C) positioned on the supporting portion 22.

The way to grasp a tubular tissue and the direction may be appropriately changed in accordance with the progress of the manipulation. Thus, sequential grafting can be efficiently performed. Further, the supporting portion 22 can also be used as a manipulation platform in an anastomosis manipulation.

As described above, the surgical holder H of the present invention is a multifunction tool which can be used for various operations by itself and which significantly improves manipulability or effectiveness during surgery.

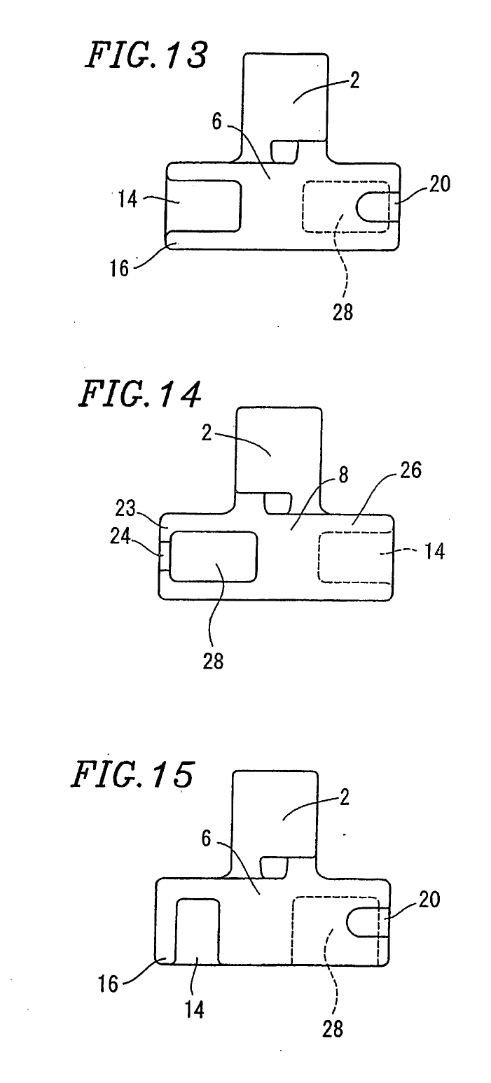

FIGS. 13 and 14 show a structure of a grasping member 1 according to another embodiment of the present invention.

In this embodiment, a second grasping plate 8 which forms part of the grasping member 1 includes a covering portion 26 formed so as to cover an entire surface or a part of an opening 14 of a first grasping plate 6, a non-covering portion 28 which does not cover the first grasping plate 6, and a fixing portion 23 having a curved portion 24. More specifically, the second grasping plate 8 includes the fixing portion 23 provided to extend from the covering portion 26, which is formed so as to cover an entire surface or a part of the opening 14 of the first grasping plate 6, through two elongated pieces, and includes the non-covering portion 28 with an opening of a rectangular shape formed by an edge of the covering portion 26, the elongated piece, and the fixing portion 23.

The first grasping plate 6 is formed with the opening 14 having a squared C-shape. Other portions of the structure are the same as those in the above embodiment.

According to the above-described structure, the curved portion 24 is formed in a part of a frame body forming the non-covering portion 28. The strength of the curved portion 24 can be enhanced.

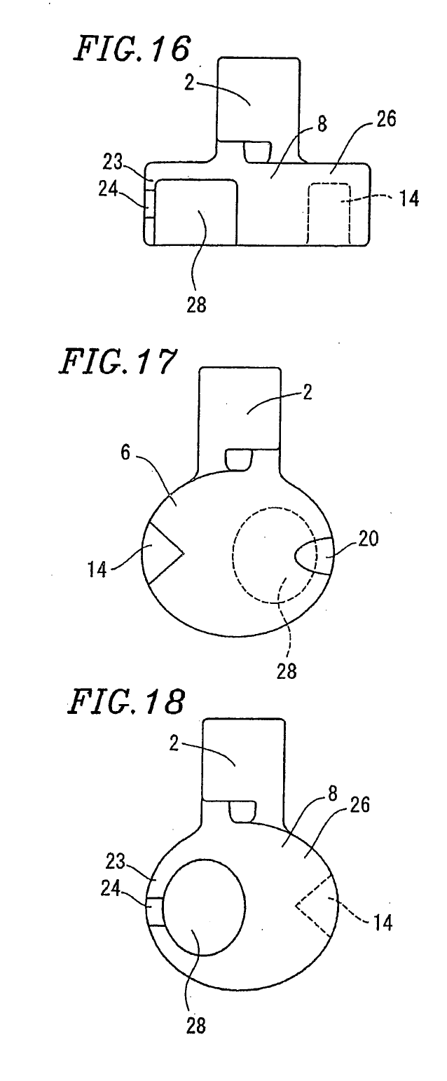

FIG. 15 and 16 show a structure of a grasping member 1 according to still another embodiment of the present invention.

In this embodiment, an opening 14 which is opened along a side edge of a first grasping plate 6, which is opposed to a manipulation member 2, is formed.

Other portions of the structure are the same as those in the above embodiment.

FIGS. 17 and 18 show a structure of a grasping member 1 according to yet another embodiment of the present invention.

In this embodiment, a first grasping plate 6 and a second grasping plate 8 which is located to oppose the first grasping plate 6 are formed into circular or elliptic shapes. An opening 14 formed at one end portion of the first grasping plate 6 is formed into a fan-shape (three-sided shape with one curved shape). A non-covering portion 28 formed in the second grasping plate 8 has a circular or elliptic shape. Other portions of the structure are the same as those in the above embodiment.

According to the above-described embodiment, the strength of the curved portion 24 or the fixing portion 23 forming the curved portion 24 can be enhanced. Further, since there are no corners in the grasping member 1, there is less fracture or damage.

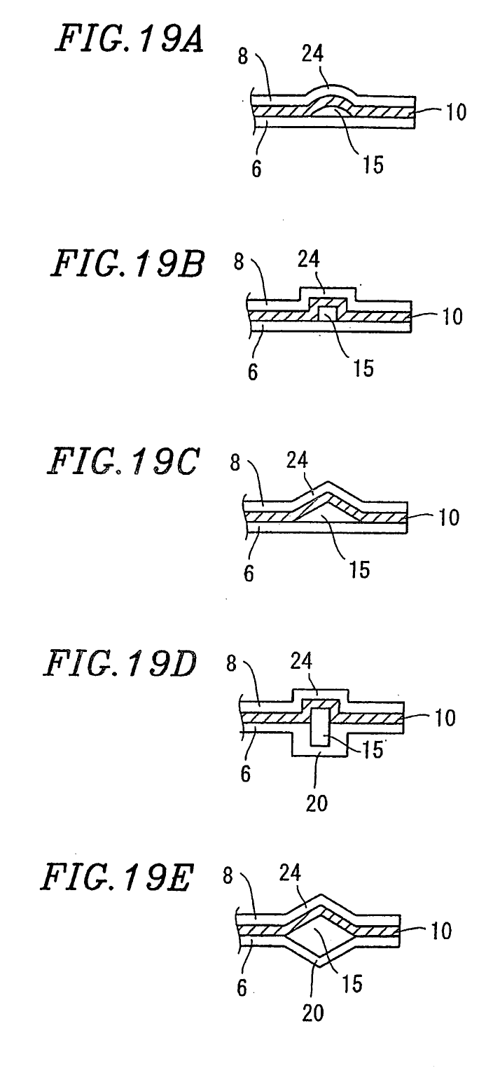

Moreover, the tissue grasping space 15 formed between the first grasping plate 6 and the second grasping plate 8 may have the structure as shown in FIGS. 19A through 19E.

FIGS. 19A through 19C show a structure in which a recessed portion or a curved portion is formed on only one of the first grasping plate 6 and the second grasping plate 8. FIG. 19A shows a structure having a curved recessed portion. FIG. 19B shows a structure with a recessed portion having a rectangular cross-section. FIG. 19C shows a structure with a recessed portion having a triangular cross-section.

FIGS. 19D and 19E show a structure with a recessed portion formed in an inner surface of the first grasping plate 6 and the second grasping plate 8. FIG. 19D shows a structure formed with a recessed portion having a rectangular cross-section. FIG. 19E shows a structure formed with a recessed portion having a triangular cross-section.

Hereinafter, Examples of the surgical holder H of the present invention will be described with respect to a manner when cardiac surgery is performed, with reference to the drawings. The present invention is not limited to these Examples.

### EXAMPLES

In the Examples, tubular tissues in the drawings are explained as an artery or a vein.

### Example 1

### Grasping of a Tissue Including a Tubular Tissue and a Surrounding Tissue

As shown in FIG. 8, the surgical holder H of the present invention is used to grasp an artery removed with the surrounding tissue as a so-called pedicle. A tissue including a tubular tissue and a surrounding tissue thereof is inserted between the first grasping plate 6 and the second grasping plate 8 from the side of the opening 14. The manipulation portion 2 manipulates the first grasping plate 6 and the second grasping plate 8 so as to overlay and grasp the tissue. At this time, the edge portion 16 of the retaining portion 18 which defines the shape of the opening 14 of the first grasping plate 6 and the covering portion 26 of the second grasping plate 8 grasps the surrounding tissue.

The covering portion 26 of the second grasping plate 8 supports a tubular tissue positioned at the opening 14. As described above, by grasping the surrounding tissue of the tubular tissue not the tubular tissue, it is possible to retain the tissue without causing damage to the tubular tissue.

### Example 2

### Grasping of a Skeletonized Tissue

As shown in FIG. 9, the surgical holder H of the present invention is used to grasp a skeletonized tissue. The recessed portion 20 of the first grasping plate 6 or the curved portion 24 of the second grasping plate 8 positions an artery. The manipulation

member 2 manipulates the first grasping plate 6 and the second grasping plate 8 so as to overlay to fix the tissue. Thus, it is possible to stably hold the skeletonized tissue

without cutting it.

### Example 3

### Grasping of a Skeletonized Tubular Tissue at a Plurality of Points

As shown in FIG. 10, the surgical holder H of the present invention is used such that, with one point of a skeletonized artery positioned at the opening 14 of the first grasping plate 6 and another point of the tubular tissue positioned at the tissue grasping space 15 formed by the recessed portion 20 of the first grasping plate 6 and the curved portion 24 of the second grasping plate 8, the manipulation member 2 manipulates the first grasping plate 6 and the second grasping plate 8 so as to overlay. The retaining portion 18 of the first grasping plate 6 and the covering portion 26 of the second grasping plate 8 grasp the tubular tissue at one point and the tissue grasping space 15 formed by the recessed portion 20 of the first grasping plate 6 and the curved portion 24 of the second grasping plate 8 grasps another point of the tissue. Thus, the tissue is grasped at at least two points. By grasping the tissue at a plurality of points as described above, it is possible to stably grasp the tissue.

### Example 4

### Grasping of a Vein

As shown in FIG. 11, the surgical holder H of the present invention is used to grasp a vein. The end portion of the part which defines a shape of the opening 14 of the first grasping plate 6 is inserted into a tube of a vein and the vein hangs suspended and is grasped. A side surface of the blood vessel can be further fixed by the covering portion 26 of the second plate 8 to grasp the vein more stably.

### Example 5

### Sequential Grafting

The surgical holder H of the present invention is used to grasp a skeletonized artery (A) by the retaining portion 18 of the first grasping plate 6 and the covering portion 26 of the second grasping plate 8 at one point and by the tissue grasping space 15 formed by the recessed portion 20 of the first grasping plate 6 and the curved portion 24 of the second grasping plate 8 at another point such that the tissue is grasped at at least two points. A side surface of the artery (A) which is positioned between the two points is incised in a direction parallel to a longitudinal direction of the blood vessel. The incised portion of the artery (A) is positioned on the supporting portion 22 of the first grasping plate.

Then, manipulation for side-to-side anastomosis is performed between an incised portion of an artery (B) and the incised portion of the artery (A) (FIG. 12A). Parachute suture is performed on both blood vessels with just a few stitches. Next, the artery (A) is removed from the grasping member 1 and adhered to the anastomotic portion with the artery (B). Then, the rest of the suturing is performed to complete the side-to-side anastomosis.

Subsequently, the surgical holder H of the present invention is used to grasp the proximate end of the artery (A) with the tissue grasping space 15 formed by the recessed portion 20 of the first grasping plate 6 and the curved portion 24 of the second grasping plate 8 and position the stump of the artery (A) on the supporting portion of the first grasping plate 6. Then, the manipulation for end-to-side anastomosis is performed between an incised portion of an artery (C) and the stump of the artery (A) (FIG. 12B). Parachute suture is performed on both blood vessels with just a few stitches. Next, the artery (A) is removed from the grasping plate and adhered to the anastomotic portion with the artery (C). Then, the rest of the suturing is performed to complete the end-to-side anastomosis.

### INDUSTRIAL APPLICABILITY

As described above, the surgical holder of the present invention can address various types of manipulations of surgical operations, and it is preferable for use in blood vessel surgery, and more particularly, cardiac blood vessel surgery.

1. A surgical holder comprising a grasping member for grasping a tissue, a manipulation member for manipulating the grasping member, and a connection portion with one end connected to the manipulation member, wherein:
the grasping member includes a first grasping plate, and a second grasping plate provided so as to oppose the first grasping plate in a movable manner so that they are able to become closer to each other or more distanced from each other;
the first grasping plate includes a retaining portion having an opening which is opened toward an outer side of the first grasping plate and a supporting portion having a recessed portion, the opening being provided in one end of the first grasping plate and the recessed portion being provided in the other end of the first grasping plate,
the second grasping plate includes a covering portion formed so as to cover an entire surface or a Dart of the opening of the first grasping plate, a non-covering portion which does not cover the first grasping plate, and a fixing portion having a curved portion, the covering portion being provided in one end of the second grasping plate and the curved portion being provided in the other end of the second grasping plate,
the curved portion opposing the recessed portion to form the tissue grasping space when the first grasping plate and the second grasping plate are Positioned so as to oppose on another,
a first grasping portion which can grasp a part of a tissue between the first grasping portion of the first grasping plate and the covering portion of the second grasping plate are provided in one end portion of the grasping member, and the opening exposing another part of the tissue when a part of a tissue is grasped by the first grasping portion; and
a second grasping portion which can form a tissue grasping space between the recessed portion of the first grasping plate and the curved portion of the second grasping plate is provided in another end portion of the grasping member.

2. A surgical holder according to claim 1, wherein a tissue protection material is attached to an opposing surface of the first grasping plate and/or the second grasping plate.

3. A surgical holder according to claim 2, wherein the tissue protection material is permeated with medicines.

4. (canceled)

5. A surgical holder comprising a grasping member for grasping a tissue, a manipulation member for manipulating the grasping member, and a connection portion with one end connected to the manipulation member and the other end provided with a fixing tool, wherein:
the grasping member includes a first grasping plate, and
a second grasping plate provided so as to oppose the first grasping plate in a movable manner so that they are able to become closer to each other or more distanced from each other;
the grasping member is formed into a rectangular shape with the manipulation member elongated from a side thereof;
a first grasping portion which can grasp a part of a tissue between the first grasping plate and the second grasping plate, and an opening which is provided in the vicinity of the first grasping portion and exposes another part of the tissue are provided in one end portion of the grasping member, the opening being opened at an outer side of the first grasping portion;
a second grasping portion which can form a tissue grasping space between the first grasping plate and the second grasping plate is provided in another end portion of the grasping member, the tissue grasping space and the opening being positioned at respective ends on one axis of the grasping member.

6. A surgical holder comprising a grasping member for grasping a tissue, a manipulation member for manipulating the grasping member, and a connection portion connected to the manipulation member, wherein:
the grasping member includes a first grasping plate and a second grasping plate provided so as to oppose the first grasping plate in a movable manner so that they are able to become closer to each other or more distanced from each other;
the first grasping plate includes a retaining portion having an opening of a U-shape or substantially a U-shape, and a supporting portion having a recessed portion, the opening being provided in one end of the first grasping plate and the recessed portion being provided in the other end of the first grasping plate; and
the second grasping plate includes a covering portion formed to cover an entire surface or a part of the opening of the first grasping plate, a non-covering portion which does not cover the first grasping plate, and a fixing portion having a curved portion, the curved portion opposing the recessed portion to form the tissue grasping space when the first grasping plate and the second grasping plate are positioned so as to oppose one another.

7. A surgical holder according to claim 6, wherein the tissue to be grasped is a tubular tissue with a surrounding tissue, and the surrounding tissue is grasped by a part which defines the shape of the opening of the first grasping plate and the covering portion of the second gasping plate.

8. A surgical holder according to claim 6, wherein the tissue to be grasped is a tubular tissue, and the tubular tissue is grasped by a tissue grasping space formed by the recessed portion of the first grasping plate and the curved portion of the second grasping plate.

9. A surgical holder according to claim 6, wherein the tissue to be grasped is a tubular tissue, and the tubular tissue is grasped with one point of the tubular tissue being grasped by the retaining portion of the first grasping plate and the covering portion of the second gasping plate, and another point being grasped by a tissue grasping space formed by the recessed portion of the first grasping plate and the curved portion of the second grasping plate.

10. A surgical holder according to claim 6, wherein the tissue to be grasped is a tubular tissue, and an end portion of the retaining portion which defines the opening of the first grasping plate is inserted into a tube of the tubular tissue to grasp the tubular tissue.

11. A surgical holder according to claim 6, wherein the tissue to be grasped is a tubular tissue (A), and, (i) with one point of the tubular tissue (A) grasped by the retaining portion of the first grasping plate and the covering portion of the second grasping plate and another point of the tubular tissue (A) grasped by the tissue grasping space formed by the recessed portion of the first grasping plate and the curved portion of the second grasping plate, anastomosis manipulation between a cut portion of the tubular tissue (A) and a cut portion of a tubular tissue (B) positioned on the supporting portion is performed, and then, (ii) with the tubular tissue (A) grasped by the tissue grasping space formed by the recessed portion of the first grasping plate and the curved portion of the second grasping plate, anastomosis manipulation between a cut portion of the tubular tissue (A) and a cut portion of the tubular tissue (C) positioned on the supporting portion is performed.
